(19) United States
(12) Patent Application Publication
(10) Pub. No.: US 2005/0169950 A1
(43) Pub. Date: Aug. 4, 2005

(54) Solid cosmetic composition comprising fibers

(76) Inventors: Marie-Laure Delacour, Paris (FR); Xavier Ray, Villeconin (FR)

Correspondence Address: Thomas L. Irving;FINNEGAN, HENDERSON, FARABOW, GARRETT & DUNNER, L.L.P., 1300 I Street, N.W., Washington, DC, 20005-3315, US

(21) Appl. No.: 11/013,540

(22) Filed: Dec. 17, 2004

Related U.S. Application Data
(63) Continuation-in-part of application No. 10/614,016, filed on Jul. 8, 2003.
(60) Provisional application No. 60/534,104, filed on Jan. 5, 2004.

(30) Foreign Application Priority Data
Dec. 19, 2003 (FR) 03 51128
Jan. 8, 2002 (WO) PCT/FR02/00044

(51) Int. Cl. A61K 7/06; A61K 7/11

(52) U.S. Cl. 424/401; 424/70.12

(57) ABSTRACT

Disclosed herein is a solid composition comprising water, solid particles of elastomeric organopolysiloxane, fibers and at least one additional powder. The composition may exhibit a high disintegration capacity without impairment of the surface appearance. The composition disclosed herein may be used for making up and caring for keratin materials, especially the skin.

This application claims benefit of U.S. Provisional Application No. 60/534,104, filed Jan. 5, 2004. This application is also a continuation-in-part of U.S. application Ser. No. 10/614,016, filed on Jul. 8, 2003, and published on Apr. 15, 2004, as U.S. Patent Application Publication No. 2004/0071648.

Disclosed herein is a solid composition, for example a cosmetic composition, comprising particles of elastomeric organopolysiloxane and fibers. Further disclosed herein is a makeup or non-therapeutic treatment process for human keratin material, such as the skin, the nails, the eyelashes, the eyebrows, and the hair, comprising applying the composition disclosed herein to keratin material.

The composition disclosed herein may be a makeup or care composition for keratin material, such as the skin.

In one embodiment, the presently disclosed composition is a makeup compositions. The makeup composition may be a skin makeup product, such as a complexion product (for example a foundation), an eyeshadow, an eyeliner, a makeup rouge, a concealer product, a body makeup product, a lip makeup product, a nail makeup product, and/or a hair makeup product. In certain embodiments, the composition is a skin makeup product, such a foundation, an eyeshadow or a makeup rouge. For example, the composition disclosed herein may relate to a complexion makeup product, such as a foundation.

The skincare composition may be a skincare product (for the face, the body and/or the hands), a skin matting product, an antisun product for the skin (such as for the face), a self-tanning composition, and/or a deodorant product.

Solid cosmetic compositions comprising particles of elastomeric crosslinked organopolysiloxane in aqueous medium and powders such as pigments and fillers are known from PCT Patent Application WO 02/053126. These compositions may have a cohesive pasty texture that is solid in form. In addition, these solid compositions may be packaged in a dish and may have a take-up surface with an elastic nature, thus affording a novel feel different from that of standard compact powders, which may feel hard and rigid.

To use the solid compositions described in PCT Patent Application WO 02/053126, the user rubs the surface of the solid product with the aid of an applicator such as a sponge, powder puff or brush or else with the finger, in order to remove the desired amount of product, which is intended for application to the keratin material to be treated or made up. Owing to the specific texture (such as flexibility and elasticity) of the product, this product may be difficult to pick up with the applicator, and, when the user rubs the surface of the product with excessive force, the surface may be impaired, by breaking up or developing cracks. The surface of the product may then become irregular and unattractive, and it may become even more difficult to remove the product. The product may no longer have an attractive appearance to the user and therefore may become adverse.

It would be desirable, therefore, to provide a solid composition comprising particles of elastomeric crosslinked organopolysiloxane and at least one additional powder, which can be taken up using an applicator or by a finger without impairing the surface of the product.

The present inventors have discovered that such a composition may be obtained by introducing fibers into the composition, wherein the fibers may make it possible to reinforce the shear strength of the surface of the product and thus to prevent impairment of the surface when the product is taken up with an applicator. In the course of its use, the product may not exhibit surface fragmentation and may not develop cracks, but may retain a regular and aesthetic surface appearance.

European Patent Application No. EP 1 064 930 discloses a cosmetic composition comprising particles of elastomeric crosslinked organopolysiloxane and fibers for the purpose of obtaining a homogeneous and uniform makeup that has a soft feel. That document, however, does not recommend employing the fibers for the purpose of reinforcing the shear strength of a solid product comprising powders.

One embodiment disclosed herein is, for example, a solid composition comprising water, solid particles of elastomeric organopolysiloxane, fibers, and at least one additional powder. The composition may, for example, be a cosmetic composition.

Another embodiment disclosed herein is a cosmetic makeup or non-therapeutic treatment process for keratin material, such as for the skin, comprising applying to the keratin material, such as the skin, a composition as disclosed herein.

As used herein, the term “solid composition” means a composition that does not flow under its own weight at room temperature (25° C.) after one hour.

The composition disclosed herein comprises particles of elastomeric crosslinked organopolysiloxane.

The elastomeric crosslinked organopolysiloxane may be obtained via a crosslinking addition reaction of diorganopolysiloxane containing at least one hydrogen linked to silicon and of diorganopolysiloxane containing ethylenically unsaturated groups linked to silicon, for example in the presence of a platinum catalyst; or via a dehydrogenation crosslinking condensation reaction between a diorganopolysiloxane containing hydroxyl end groups and a diorganopolysiloxane containing at least one hydrogen linked to silicon, for example in the presence of an organotin; or via a crosslinking condensation reaction of a diorganopolysiloxane containing hydroxyl end groups and of a hydrolysable organopolysilane; or via thermal crosslinking of organopolysiloxane, for example in the presence of an organoperoxide catalyst; or via crosslinking of organopolysiloxane by high-energy radiation such as gamma rays, ultraviolet rays, and/or an electron beam.

In certain embodiments, the elastomeric crosslinked organopolysiloxane is obtained via a crosslinking addition reaction of (A) diorganopolysiloxane containing at least two hydrogens each linked to a silicon, and (B) diorganopolysiloxane containing at least two ethylenically unsaturated groups (such as vinyl groups) linked to silicon, for example in the presence of (C) a platinum catalyst, for instance as described in European Patent Application No. EP A 295 886.

For example, the organopolysiloxane may be obtained via reaction of dimethylpolysiloxane containing dimethylvinylsiloxy end groups and of methyl-hydrogenopolysiloxane containing trimethylsiloxy end groups, in the presence of a platinum catalyst.

In certain embodiments, compound (A) is the base reagent for the formation of elastomeric organopolysiloxane, and the crosslinking takes place via an addition reaction of compound (A) with compound (B) in the presence of the catalyst (C).

Compound (A) may be an organopolysiloxane containing at least two hydrogen atoms linked to different silicon atoms in each molecule.

Compound (A) may have any molecular structure, such as a linear-chain, branched-chain, or cyclic structure.

Compound (A) may have a viscosity at 25° C. ranging from 1 to 50,000 centistokes, which may show good miscibility with compound (B).

The organic groups linked to the silicon atoms in compound (A) may be chosen from alkyl groups such as methyl, ethyl, propyl, butyl, and octyl; substituted alkyl groups such as 2-phenylethyl, 2-phenylpropyl, and 3,3,3-trifluoropropyl; aryl groups such as phenyl, tolyl, and xylyl; substituted aryl groups such as phenylethyl; and substituted monovalent hydrocarbon-based groups such as epoxy groups, carboxylate ester groups, and mercapto groups.

Compound (A) may thus be chosen from methylhydrogenopolysiloxanes containing trimethylsiloxy end groups, dimethylsiloxane-methylhydrogenosiloxane copolymers containing trimethylsiloxy end groups, and dimethylsiloxane-methylhydrogenosiloxane cyclic copolymers.

Compound (B) may be a diorganopolysiloxane containing at least two lower alkenyl groups (for example C₂-C₄). The lower alkenyl group may be chosen from vinyl, allyl, and propenyl groups. These lower alkenyl groups may be located in any position on the organopolysiloxane molecule, for example they may be located at the end of the organopolysiloxane molecule. The organopolysiloxane (B) may have a branched-chain, linear-chain, cyclic, or network structure, for example a linear-chain structure. Compound (B) may have a viscosity ranging from the liquid state to the gum state. For example, compound (B) may have a viscosity of at least 100 centistokes at 25° C.

The organopolysiloxanes (B) may be chosen from methylvinylsiloxanes, methylvinylsiloxane-dimethylsiloxane copolymers, dimethylpolysiloxanes containing dimethylvinylsiloxy end groups, dimethylsiloxane-methylphenylsiloxane copolymers containing dimethylvinylsiloxy end groups, dimethylsiloxane-diphenylsiloxane-methylvinylsiloxane copolymers containing dimethylvinylsiloxy end groups, dimethyl-siloxane-methylvinylsiloxane copolymers containing trimethylsiloxy end groups, dimethylsiloxane-methylphenylsiloxane-methylvinylsiloxane copolymers containing trimethylsiloxy end groups, methyl(3,3,3-trifluoropropyl)polysiloxane containing dimethylvinylsiloxy end groups, and dimethylsiloxane-methyl(3,3,3-trifluoropropyl)siloxane copolymers containing dimethylvinylsiloxy end groups.

Besides the above-mentioned alkenyl groups, the other organic groups linked to the silicon atoms in compound (B) may be chosen from alkyl groups such as methyl, ethyl, propyl, butyl, and octyl; substituted alkyl groups such as 2-phenylethyl, 2-phenylpropyl, and 3,3,3-trifluoropropyl; aryl groups such as phenyl, tolyl, and xylyl; substituted aryl groups such as phenylethyl; and substituted monovalent hydrocarbon-based groups such as epoxy groups, carboxylate ester groups, and mercapto groups.

For example, the elastomeric organopolysiloxane may be obtained via reaction of dimethylpolysiloxane containing dimethylvinylsiloxy end groups and of methylhydrogenopolysiloxane containing trimethylsiloxy end groups, in the presence of a platinum catalyst.

In certain embodiments, the sum of the number of ethylenic groups per molecule of compound (B) and of the number of hydrogen atoms linked to silicon atoms per molecule of compound (A) is at least 5.

Compound (A) may be added in an amount such that the molecular ratio between the total amount of hydrogen atoms linked to silicon atoms in compound (A) and the total amount of all the ethylenically unsaturated groups in compound (B) ranges from 1.5/1 to 20/1.

Compound (C) is the catalyst for the crosslinking reaction, and may, for example, be chosen from chloroplatinic acid, chloroplatinic acid-olefin complexes, chloro-platinic acid-alkenylsiloxane complexes, chloroplatinic acid-diketone complexes, platinum black, and platinum on a support.

The catalyst (C) may be added in a proportion ranging from 0.1 to 1,000 parts by weight, such as from 1 to 100 parts by weight, as clean platinum metal per 1,000 parts by weight of the total amount of compounds (A) and (B).

Such particles of elastomeric crosslinked organopolysiloxane are, for example, described in Japanese Patent Application No. JP A 61 194 009 and European Patent Application Nos. EP A 242 219 and EP A 381 166.

In certain embodiments, the elastomer is a non-emulsifying elastomer. As used herein, the term “non-emulsifying” defines organopolysiloxane elastomers containing no polyoxyalkylene units.

Elastomers in powder form that may be used include those sold under the names DC 9505 and DC 9506 by the company Dow Corning.

According to one embodiment disclosed herein, the particles of elastomeric crosslinked organopolysiloxane used may be in the form of an aqueous dispersion.

The elastomeric organopolysiloxanes disclosed herein may be chosen from the crosslinked polymers, such as those described in Japanese Patent Application No. JP A 10/175,816 and U.S. Pat. No. 5,928,660. According to this Japanese patent application, the elastomeric organopolysiloxanes are obtained via a crosslinking addition reaction, in the presence of a catalyst, such as a platinum type catalyst, of at least:
(a) one organopolysiloxane (i) containing at least two vinyl groups in a-w position on the silicone chain per molecule; and
(b) one organosiloxane (ii) containing at least one hydrogen atom linked to a silicon atom per molecule.

For example, the organopolysiloxane (i) may be chosen from polydimethylsiloxanes and may be an α-ω-dimethylvinyl polydimethylsiloxane.

The elastomeric organopolysiloxanes in the composition disclosed herein may be in the form of an aqueous suspension. This suspension may be obtained as follows:
(a) mixing an organopolysiloxane (i) containing at least two vinyl groups in α-ω position on the silicone chain per molecule and an organosiloxane (ii) containing at least one hydrogen atom linked to a silicon atom per molecule;
(b) adding a catalyst, such as a platinum type catalyst;
(b) adding an aqueous phase comprising at least one emulsifier to form an emulsion;
(c) emulsion polymerizing the organopolysiloxane (i) and the organosiloxane (ii) in the presence of a platinum catalyst.

The emulsifier may be chosen from nonionic, cationic and anionic surfactants with an HLB greater than or equal to 8, and may be, for example, nonionic surfactants.

The proportion of surfactants may range from 0.1 to 20 parts by weight, such as from 0.5 to 10 parts by weight per 100 parts by weight of the elastomeric organopolysiloxane composition (compare, for example, the description of Japanese Patent Application No. JP A 10/175,816).

After step (c), it is possible to dry the particles obtained and to evaporate therefrom all or some of the trapped water.

The organopolysiloxanes may be in the form of deformable solid particles having a certain hardness, which may be measured using a Shore A durometer (according to ASTM standard D2240) at room temperature or via Japanese method JIS-A. This hardness may be measured on an elastomeric block prepared for this purpose as follows: mixing the organopolysiloxane (i) and the organosiloxane (ii); removing air from the mixture; molding and vulcanizing in an oven at 100° C. for 30 minutes; cooling to room temperature, and measuring the hardness. The density may also be determined on this block of elastomer.

The organopolysiloxane may have a JIS-A hardness of less than or equal to 80, such as ranging from 10 to 80, or for example less than or equal to 65, such ranging from 15 to 65.

As organopolysiloxane particles dispersed in water, it is possible to use those sold under the names BY 29-122 and BY 29-119 by the company Dow Corning.

The particles of elastomeric crosslinked organopolysiloxane may be present in the composition disclosed herein in an amount ranging from 10% to 50% by weight, such as ranging from 20% to 40% by weight, or ranging from 25% to 40% by weight, relative to the total weight of the composition.

The composition disclosed herein also comprises fibers.

As used herein, the term “fiber” should be understood as meaning an object of length L and diameter D such that L is greater than D, D being the diameter of the circle in which the cross section of the fiber is inscribed. For example, the ratio LID (or shape factor) may range from 3.5 to 2,500, such as from 5 to 500 or from 5 to 150.

The fibers that may be used in the composition disclosed herein may be chosen from mineral and organic fibers of synthetic or natural origin. They may be short or long and individual or organized, for example braided, and they may be hollow or solid. They may have any shape, for example they may have a circular or polygonal (square, hexagonal, or octagonal) cross section, depending on the intended specific application. In certain embodiments, their ends are blunt and/or polished to prevent injury.

For example, the fibers may have a length ranging from 1 μm to 10 mm, such as from 0.1 mm to 5 mm or from 0.3 mm to 1 mm. Their cross section may be within a circle having a diameter ranging from 2 nm to 500 μm, such as ranging from 100 nm to 100 μm or from 1 μm to 50 μm. The weight or yarn count of the fibers may be given in denier or decitex, and represents the weight in grams per 9 km of yarn. For exarriple, the fibers disclosed herein may have a yarn count ranging from 0.01 to 10 denier, such as from 0.1 to 2 denier or from 0.3 to 0.7 denier.

The fibers may be those used in the manufacture of textiles, such as silk fibers; cotton fibers; wool fibers; flax fibers; cellulose fibers extracted, for example, from wood, from plants or from algae; rayon fibers; polyamide (Nylon®) fibers; viscose fibers; acetate fibers, such as rayon acetate fibers; poly(p-phenyleneterephthalamide) (or aramide) fibers, such as Kevlar® fibers; acrylic polymer fibers, such as polymethyl methacrylate fibers and poly(2-hydroxyethyl methacrylate) fibers; polyolefin fibers; polyethylene fibers; polypropylene fibers; glass fibers; silica fibers; carbon fibers, such as in graphite form; polytetrafluoroethylene (such as Teflon®) fibers; insoluble collagen fibers; polyester fibers; polyvinyl chloride fibers; polyvinylidene chloride fibers; polyvinyl alcohol fibers; polyacrylonitrile fibers; chitosan fibers; polyurethane fibers; polyethylene phthalate fibers; and mixtures thereof, for instance polyamide/polyester fibers.

In one embodiment, the fibers are polyamide (Nylon®) fibers.

The fibers used in surgery may also be used, for instance the resorbable synthetic fibers prepared from glycolic acid and caprolactone (for example, Monocryl® from Johnson & Johnson); resorbable synthetic fibers of the type which are copolymers of lactic acid and of glycolic acid (for example, Vicryl® from Johnson & Johnson); polyterephthalic ester fibers (for example, Ethibond® from Johnson & Johnson); and stainless steel threads (for example, Acier® from Johnson & Johnson).

Moreover, the fibers may be treated or untreated at the surface, and uncoated or coated with a protective layer.

As coated fibers that may be used, mention may be made of polyamide fibers coated with copper sulphide to give an anti-static effect (for example R-STAT from Rhodia) or another polymer allowing a special organization of fibers (treatment of the specific surface). Mention may also be made of fibers coated with mineral or organic pigments, such as the pigments mentioned below.

In some embodiments, fibers of synthetic origin and organic fibers, such as those used in surgery, may be used.

The fibers that may be used in the composition according to the present disclosure may be chosen from at least one of polyamide fibers, cellulose fibers, poly(p-phenyleneterephthalamide) fibers, and polyethylene fibers. Their length (L) may range from 0.1 mm to 5 mm, such as from 0.25 mm to 1.6 mm, and their mean diameter may range from 1 μm to 50 μm. In certain embodiments, the polyamide fibers sold by Etablissements P. Bonte under the name Polyamide 0.9 Dtex, having a mean diameter of 6. μm, a yarn count of 0.9 decitex, and a length ranging from 0.3 mm to 5 mm, may be used, or the polyamide fibers sold under the name of Fiberlon 931-D1-S by the company LCW, having a yarn count of 0.9 decitex and a length of 0.3 mm may be used. Cellulose (or rayon) fibers with a mean diameter of 50 μm and a length ranging from 0.5 mm to 6 mm may also be used, for instance those sold under the name Natural rayon flock fiber RC1BE -N003-M04 by the company Claremont Flock. Polyethylene fibers, for instance those sold under the name Shurt Stuff 13 099 F by the company Mini Fibers, may also be used.

The composition disclosed herein may also comprise “rigid” fibers, as opposed to the fibers mentioned above, which are not rigid fibers.

The rigid fibers, which are initially substantially straight, when placed in a dispersing medium, do not undergo a substantial change in shape, which is reflected by the angular condition defined below, reflecting a shape that may be described as still substantially straight and linear. This angle condition reflects the stiffness of the fibers, which may be difficult to express by another parameter for objects that are as small as the rigid fibers.

The stiffness of the fibers may be reflected by the following angular condition: in certain embodiments, at least 50% numerically, for example at least 75% numerically or at least 90% numerically, of the fibers are such that the angle formed between the tangent to the longitudinal central axis of the fiber and the straight line connecting the end of the fiber to the point on the longitudinal central axis of the fiber corresponding to half the length of the fiber is less than 15°, and the angle formed between the tangent to the longitudinal central axis of the fiber at a point half way along the fiber and the straight line connecting one of the ends to the point on the longitudinal central axis of the fiber corresponding to half the length of the fiber, is less than or equal to 150 for the same fiber length ranging from 0.8 mm to 5 mm, for example ranging from 1 mm to 4 mm or ranging from 1 mm to 3 mm, or having a fiber length of 2 mm.

In certain embodiments, the angle mentioned above is measured at the two ends of the fiber and at a point half way along the fiber; in other words, three measurements are taken in this case, and the average of the measured angles is less than or equal to 15°.

The tangent, at any point on the fiber may form an angle of less than 15°.

In the present disclosure, the angle formed by the tangent at a point on the fiber is the angle formed between the tangent to the longitudinal central axis of the fiber at the said point on the fiber and the straight line connecting the end of the fiber that is closest from the said point to the point on the longitudinal central axis of the fiber corresponding to half the length of the fiber.

Generally, the rigid fibers that may be used in the composition disclosed herein may have the same or substantially the same fiber length.

More specifically, when a medium in which the rigid fibers are dispersed to a fiber concentration of 1% by weight is observed by microscope, with an objective lens allowing a magnification of 2.5 and with full-field vision, a numerical majority of the rigid fibers, i.e., at least 50% numerically of the rigid fibers, for example at least 75% numerically of the rigid fibers or at least 90% numerically of the rigid fibers, should satisfy the angular condition defined above. The measurement leading to the angle value is performed for the same length of fibers, this length ranging from 0.8 mm to 5 mm, such as from 1 to 4 mm or from 1 to 3 mm, of having a length of 2 mm.

The medium in which the observation is performed is a dispersing medium that ensures good dispersion of the rigid fibers, for example water or an aqueous gel of clay or of associative polyurethane. A direct observation of the composition comprising the rigid fibers may even be performed. A sample of the prepared composition or dispersion may be placed between a slide and cover slip for observation by microscope with an objective lens allowing a magnification of 2.5 and with full-field vision. Full-field vision may allow the fibers to be viewed in their entirety.

The rigid fibers may be chosen from fibers of a synthetic polymer chosen from polyesters, polyurethanes, acrylic polymers, polyolefins, polyamides such as non-aromatic polyamides, and aromatic polyimideamides.

Examples of rigid fibers that may be mentioned include:
polyester fibers, such as those obtained by chopping yarns sold under the names Fibre 255-100-R 1-242T Taille 3 mm (eight-lobed cross section), Fibre 265-34-R 1-56T Taille 3 mm (round cross section), and Fibre Coolmax 50-34-591 Taille 3 mm (four-lobed cross section) by the company Dupont de Nemours;
polyamide fibers, such as those sold under the names Trilobal Nylon 0.120-1.8 DPF; Trilobal Nylon 0.120-18 DPF; Nylon 0.120-6 DPF by the company Cellusuede Products; or polyamide fibers obtained by chopping yarns sold under the name Fibre Nomex® Brand 430 Taille 3 mm by the company DuPont de Nemours;
polyimideamide fibers, such as those sold under the names Kermel® and Kermel® Tech by the company Rhodia;
poly(p-phenyleneterephthalamide) (or aramide) sold for example under the name Kevlar® by the company Dupont de Nemours;
fibers with a multilayer structure comprising alternating layers of polymers chosen from polyesters, acrylic polymers, and polyamides, such as those described in European Patent Application Nos. EP A 6 921 217 and EP A 686 858 and U.S. Pat. No. 5,472,798. Such fibers are sold under the names Morphotex® and Teijin Tetron Morphotex® by the company Teijin.

Rigid fibers that may be used include aromatic polyimideamide fibers.

Polyimideamide yarns or fibers that may be used for the compositions disclosed herein are described, for example, in the document from R. Pigeon and P. Allard, Chimie Macromoléculaire Appliquée, 40/41 (1974), pages 139-158 (No. 600), U.S. Pat. No. 3,802,841, French Patent No. FR A 2 079 785, and European Patent Application Nos. EP A1 0 360 728 and EP A 0 549 494, all of which are incorporated by reference herein.

Aromatic polyimideamide fibers that may be used according to certain embodiments are polyimideamide fibers comprising repeating units of formula: 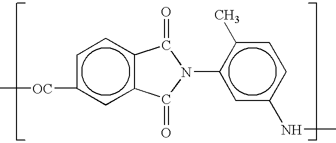
obtained by polycondensation of tolylene diisocyanate and trimellitic anhydride.

The fibers may be present in the composition disclosed herein in an amount ranging from 0.5% to 0.20% by weight, such as from 0.5% to 15% by weight, from 0.5% to 8% by weight, or from 2% to 8% by weight, relative to the total weight of the composition.

In certain embodiments, the fibers and the particles of elastomeric crosslinked organopolysiloxane are present in the composition in amounts such that the fibers/particles of crosslinked organopolysiloxane weight ratio ranges from 0.01 to 2, such as from 0.03 to 1, from 0.05 to 0.5, or from 0.05 to 0.2.

The composition disclosed herein comprises at least one additional powder different from the particles of elastomeric crosslinked organopolysiloxane and the fibers described above.

The additional powder may be chosen from pulverulent dyestuffs, fillers, and mixtures thereof.

The pulverulent dyestuff may be chosen from pigments, nacres, and mixtures thereof.

As used herein, the term “pigments” should be understood as meaning white or colored, mineral or organic particles of any form, which are insoluble in the physiological medium, and are intended to color the composition.

As used herein, the term “nacres” should be understood as meaning iridescent particles of any form, for example particles produced by certain molluscs in their shell or particles alternatively synthesized.

The pigments may be white or colored and mineral and/or organic. Among the mineral pigments that may be mentioned are titanium dioxide, optionally surface-treated, zirconium oxides, cerium oxides, zinc oxides, iron oxides (black, yellow and red), chromium oxides, manganese violet, ultramarine blue, chromium hydrate, ferric blue, and metal powders such as aluminium powder and copper powder.

Among the organic pigments that may be mentioned are carbon black, b & C pigments, and lakes based on cochineal carmine, barium, strontium, calcium, or aluminium.

The nacreous pigments may be chosen from white nacreous pigments such as mica coated with titanium or with bismuth oxychloride; colored nacreous pigments such as titanium mica with iron oxides; titanium mica with, for example, ferric blue or chromium oxide; titanium mica with an organic pigment of the abovementioned type; and nacreous pigments based on bismuth oxychloride.

The pulverulent dyestuffs may be present in the composition disclosed herein in an amount ranging from 0.5% to 60% by weight, such as ranging from 0.5% to 50% by weight, ranging from 0.5% to 0.40% by weight, ranging from 1% to 30% by weight, or ranging from 3% to 25% by weight, relative to the total weight of the composition.

The composition disclosed herein may further comprise at least one filler.

As used herein, the term “filler” should be understood as meaning colorless or white, mineral or synthetic particles of any form, which are insoluble in the medium of the composition, irrespective of the temperature at which the composition is manufactured.

The at least one filler may be mineral or organic, of any form, platelet-form, spherical or oblong, irrespective of the crystallographic form (for example laminar, cubic, hexagonal, orthorhombic, etc.). Mention may be made of talc, mica, silica, kaolin, polyamide (Nylon®) powder, poly-β-alanine powder, polyethylene powder, tetrafluoroethylene polymer (Teflon®) powder, lauroyllysine, starch, boron nitride, hollow polymer microspheres such as those of polyvinylidene chloride/acrylonitrile, for instance Expancel® (Nobel Industrie) and of acrylic acid copolymers, ethylene glycol dimethacrylate and lauryl methacrylate copolymer powders, for example those sold under the name Polytrap® 6603 Adsorber by the company RP Scherer, silicone resin microbeads (Tospearls® from Toshiba, for example), precipitated calcium carbonate, magnesium carbonate, magnesium hydrocarbonate, hydroxyapatite, hollow silica microspheres, glass microcapsules, ceramic microcapsules, metal soaps derived from organic carboxylic acids containing from 8 to 22 carbon atoms, such as 12 to 18 carbon atoms, for example zinc stearate, magnesium stearate, and lithium stearate, zinc laurate, magnesium myristate, and mixtures thereof.

In certain embodiments, the at least one filler is chosen from mica, polyamide powders, ethylene glycol dimethacrylate and lauryl methacrylate copolymer powders, boron nitride, and mixtures thereof.

The at least one filler may be present in the composition in an amount ranging from 0.5% to 40% by weight, such as ranging from 1% to 35% by weight or ranging from 5% to 30% by weight, relative to the total weight of the composition.

In certain embodiments, the composition disclosed herein may comprise a total content of additional powders (including dyestuff and at least one filler) ranging from 10% to 70% by weight, such as from 10% to 60% by weight, ranging from 10% to 60% by weight, or ranging from 20% to 50% by weight, relative to the total weight of the composition.

In certain embodiments, the particles of elastomeric crosslinked organopolysiloxane and the additional particles are present in the composition disclosed herein in amounts such that the particles of crosslinked organopolysiloxane/additional powder weight ratio ranges from 0.4/1 to 2.5/1, such as from 0.4/1 to 2/1, from 0.4/1 to 1.5/1, from 0.4/1 to 1.3/1, or from 0.6/1 to 1.3/1.

The composition disclosed herein may further comprise water. The water may be a floral water such as cornflower water and/or a mineral water, such as Vittel®, eau de Lucas, and eau de La Roche Posay, and/or a spring water.

The composition may comprise water in an amount ranging from 5% to 30% by weight, such as ranging from 10% to 25% by weight, ranging from 15% to 25% by weight, or ranging from 20% to 25% by weight, relative to the total weight of the composition.

The composition may also comprise at least one polyol, for example at least one polyol containing from 2 to 20 carbon atoms, such as containing from 2 to 10 carbon atoms or containing from 2 to 6 carbon atoms. Polyols that may be mentioned include glycerol, propylene glycol, butylene glycol, pentylene glycol, hexylene glycol, dipropylene glycol, diethylene glycol, and mixtures thereof.

The composition disclosed herein may comprise at least one polyol in an amount ranging from 1% to 20% by weight, such as from 3% to 15% by weight, relative to the total weight of the composition.

The composition may comprise at least one other cosmetic ingredient, which may be chosen, for example, from antioxidants, fragrances, preserving agents, neutralizers, surfactants, waxes, oils, sunscreens, vitamins, moisturizers, self-tanning compounds, and antiwrinkle active agents.

Needless to say, a person skilled in the art will take care to select this at least one othercosmetic ingredient, and/or the amount thereof, such that the advantageous properties of the composition disclosed herein are not, or are not substantially, adversely affected by the envisaged addition.

In certain embodiments, the composition disclosed herein may have a hardness ranging from 0.07 N to 0.4 N, such as from 0.1 to 0.35 N, and an elasticity EL ranging from 15% to 80%, such as an elasticity EL ranging from 30% to 70%.

The hardness and the elasticity of the composition may be measured at 20° C. using the texturometer sold under the name TA-XT2i by the company Rheo, equipped with a stainless-steel spindle in the form of a bead 12.7 mm in diameter, by measuring the change in force (compression force or stretching force) (F) as a function of time, during the following operation:

The spindle is displaced at a speed of 0.1 mm/s and then penetrates into the product to a penetration depth of 0.3 mm. When the spindle has penetrated into the product to a depth of 0.3 mm, the spindle is removed at a speed of 0.1 mm/s. During the withdrawal of the spindle, the force (compression force) decreases greatly until it becomes zero after a time t. During the operation, the spindle effects a to-and-fro motion in 6 seconds.

The hardness corresponds to the maximum compression force measured during the operation; it may be expressed in Newtons.

The elasticity EL, expressed as a percentage, is determined via the relationship:
EL(%)=100×(t−3)/(6−3)

The composition disclosed herein may be prepared by mixing together the various ingredients, for example either in a Baker-Perkins turbomixer/granulator or in a continuous twin-screw blender, such as the BC21 extruder-blender from the company Clextral.

The composition may be packed in a dish or a case by pressing the mixture of ingredients.

Certain embodiments of the composition as disclosed herein are illustrated in greater detail by the examples described below.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result.

### EXAMPLE 1

A complexion makeup product having the composition below was prepared:

| | |
|---|---|
| Aqueous dispersion of particles of crosslinked | 59 g, i.e., |
| polydimethylsiloxane containing 63% by weight of | 37.1 g AM |
| crosslinked polymer (BY 29-119 from Dow Corning) | |
| Glycerol | 5 g |
| Propylene glycol | 5 g |
| Preserving agents | 1 g |
| Nylon powder (Orgasol ® 2002 Extra D Nat Cos | 10 g |
| from Atofina) | |
| Ethylene glycol dimethacrylate and lauryl methacrylate | 5 g |
| copolymer powder sold under the name Polytrap ® 6603 | |
| Adsorber by the company RP Scherer | |
| 0.9 Dtex polyamide fibers 0.3 mm long, from the company | 5 g |
| Paul Bonte | |
| Pigments (iron oxides, titanium dioxide) | 10 g |

After mixing together the ingredients, the product was packaged in a dish by pressing. The product was taken up from the surface by means of a sponge, without impairing the surface of the product.

The product obtained had a hardness of 0.2 N and an elasticity of 55%, measured according to the conditions described above.

### EXAMPLE 2

A skin matting product having the composition below was prepared:

| | |
|---|---|
| Particles of crosslinked polydimethylsiloxane as an aqueous | 59 g, i.e. |
| dispersion containing 63% by weight of crosslinked | 37.1 g AM |
| polymer (BY 29-119 from Dow Corning) | |
| Glycerol | 5 g |
| Propylene glycol | 5 g |
| Preserving agents | 1 g |
| Nylon powder (Orgasol ® 2002 Extra D Nat Cos | 20 g |
| from Atofina) | |
| Ethylene glycol dimethacrylate and lauryl methacrylate | 5 g |
| copolymer powder sold under the name Polytrap ® 6603 | |
| Adsorber by the company RP Scherer | |
| 0.9 Dtex polyamide fibers 0.3 mm long, from the company | 5 g |
| Paul Bonte | |

After mixing together the ingredients, the product was packaged in a dish by pressing. The product was taken up from the surface by means of a sponge, without impairing the surface of the product.

### EXAMPLE 3

An eyeshadow product having the composition below was prepared:

| | |
|---|---|
| Crosslinked polydimethylsiloxane particles (DC 9506 from | 28 g |
| Dow Corning) | |
| Water | 17 g |
| Glycerol | 5 g |
| Preserving agents | 1 g |
| 0.9 Dtex polyamide fibers 0.3 mm long, from the company | 4 g |
| Paul Bonte | |
| Nacres | 45 g |

After mixing together the ingredients, the product was packaged in a dish by pressing. The product was taken up from the surface by means of a sponge, without impairing the surface of the product.

### EXAMPLE 4

A complexion makeup product having the composition below was prepared:

| | |
|---|---|
| Particles of crosslinked polydimethylsiloxane as an aqueous | 55 g, i.e. |
| dispersion containing 63% by weight of crosslinked | 34.65 g AM |
| polymer (BY 29-119 from Dow Corning) | |
| Glycerol | 10 g |
| Preserving agents | 1 g |
| Nylon powder (Orgasol ® 2002 Extra D Nat Cos | 19 g |
| from Atofina) | |
| 0.9 Dtex polyamide fibers 0.3 mm long, from the company | 5 g |
| Paul Bonte | |
| Pigments (iron oxides, titanium dioxide) | 10 g |

After mixing together the ingredients, the product was packaged in a dish by pressing. The product was taken up from the surface by means of a sponge, without impairing the surface of the product.

### EXAMPLE 5

A skin matting product having the composition below was prepared:

| | |
|---|---|
| Particles of crosslinked polydimethylsiloxane as an aqueous | 55 g, i.e. |
| dispersion containing 63% by weight of crosslinked | 34.65 g AM |
| polymer (BY 29-119 from Dow Corning) | |
| Glycerol | 10 g |
| Preserving agents | 1 g |
| Nylon powder (Orgasol ® 2002 Extra D Nat Cos | 29 g |
| from Atofina) | |
| 0.9 Dtex polyamide fibers 0.3 mm long, from the company | 5 g |
| Paul Bonte | |

After mixing together the ingredients, the product was packaged in a dish by pressing. The product was taken up from the surface by means of a sponge, without impairing the surface of the product.

### EXAMPLE 6

A complexion makeup product having the composition below was prepared:

| | |
|---|---|
| Crosslinked polydimethylsiloxane particles (DC 9506 from | 40 g |
| Dow Corning) | |
| Water | 19 g |
| Glycerol | 5 g |
| Propylene glycol | 5 g |
| Preserving agents | 1 g |
| Nylon powder (Orgasol ® 2002 Extra D Nat Cos from Atofina) | 10 g |
| Ethylene glycol dimethacrylate and lauryl methacrylate | 5 g |
| copolymer powder sold under the name Polytrap ® 6603 | |
| Adsorber by the company RP Scherer | |
| 0.9 Dtex polyamide fibers 0.3 mm long, from the company | 5 g |
| Paul Bonte | |
| Pigments (iron oxides, titanium dioxide) | 10 g |

After mixing together the ingredients, the product was packaged in a dish by pressing. The product was taken up from the surface by means of a sponge, without impairing the surface of the product.

### EXAMPLE 7

A skin matting product having the composition below was prepared:

| | |
|---|---|
| Crosslinked polydimethylsiloxane particles (DC 9506 from | 40 g |
| Dow Corning) | |
| Water | 19 g |
| Glycerol | 5 g |
| Propylene glycol | 5 g |
| Preserving agents | 1 g |
| Nylon powder (Orgasol ® 2002 Extra D Nat Cos from Atofina) | 20 g |
| Ethylene glycol dimethacrylate and lauryl methacrylate | 5 g |
| copolymer powder sold under the name Polytrap ® 6603 | |
| Adsorber by the company RP Scherer | |
| 0.9 Dtex polyamide fibers 0.3 mm long, from the company | 5 g |
| Paul Bonte | |

After mixing together the ingredients, the product was packaged in a dish by pressing. The product was taken up from the surface by means of a sponge, without impairing the surface of the product.

1. A solid composition comprising water, solid particles of elastomeric crosslinked organopolysiloxane, fibers, and at least one additional powder.

2. The composition according to claim 1, wherein the crosslinked organopolysiloxane is chosen from crosslinked organopolysiloxanes obtained by at least one reaction chosen from:
a crosslinking addition reaction of diorganosiloxane containing at least one hydrogen linked to silicon and of diorganopplysiloxane containing ethylenically unsaturated groups linked to silicon;
a dehydrogenation crosslinking condensation reaction between a diorganopoly-siloxane containing hydroxyl end groups and a diorganopolysiloxane containing at least one hydrogen linked to silicon;
a crosslinking condensation reaction of a diorganopolysiloxane containing hydroxyl end groups and of a hydrolysable organopolysilane;
thermal crosslinking of organopolysiloxane; and
crosslinking of organopolysiloxane by high-energy radiation.

3. The composition according to claim 1, wherein the crosslinked organopolysiloxane is obtained via a crosslinking addition reaction of
(A) diorganopolysiloxane containing at least two hydrogens each linked to a silicon, and
(B) diorganopolysiloxane containing at least two ethylenically unsaturated groups linked to silicon.

4. The composition according to claim 3, wherein the at least two ethylenically unsaturated groups are vinyl groups.

5. The composition according to claim 3, wherein the crosslinking addition reaction occurs in the presence of a platinum catalyst.

6. The composition according to claim 1, wherein the crosslinked organopolysiloxane is obtained by reacting dimethylpolysiloxane containing dimethylvinylsiloxy end groups and methylhydrogenopolysiloxane containing trimethylsiloxy end groups, in the presence of a platinum catalyst.

7. The composition according to claim 1, wherein the particles of elastomeric crosslinked organopolysiloxane are present in an amount ranging from 10% to 50% by weight, relative to the total weight of the composition.

8. The composition according to claim 7, wherein the particles of elastomeric crosslinked organopolysiloxane are present in an amount ranging from 20% to 40% by weight, relative to the total weight of the composition.

9. The composition according to claim 8, wherein the particles of elastomeric crosslinked organopolysiloxane are present in an amount ranging from 25% to 40% by weight, relative to the total weight of the composition.

10. The composition according to claim 1, wherein the fibers are chosen from at least one of silk fibers, cotton fibers, wool fibers, flax fibers, cellulose fibers, polyamide fibers, viscose fibers, acetate fibers, poly(p-phenyleneterephthalamide) fibers, acrylic fibers, polyolefin fibers, silica fibers, carbon fibers, polytetrafluoroethylene fibers, insoluble collagen fibers, polyester fibers, polyvinyl chloride fibers, polyvinylidene chloride fibers, polyvinyl alcohol fibers, polyacrylonitrile fibers, chitosan fibers, polyurethane fibers, polyethylene phthalate fibers, fibers formed from a mixture of polymers, and substantially rectilinear rigid fibers.

11. The composition according to claim 10, wherein the acetate fibers are chosen from rayon acetate fibers.

12. The composition according to claim 10, wherein the acrylic fibers are chosen from at least one of polymethyl methacrylate fibers and poly(2-hydroxy methacrylate) fibers.

13. The composition according to claim 10, wherein the polyolefin fibers are chosen from at least one of polyethylene fibers and polypropylene fibers.

14. The composition according to claim 10, wherein the carbon fibers are in graphite form.

15. The composition according to claim 1, wherein the fibers are fibers of synthetic origin.

16. The composition according to claim 1, wherein the fibers have a length ranging from 1 μm to 10 mm.

17. The composition according to claim 16, wherein the fibers have a length ranging from 0.1 mm to 5 mm.

18. The composition according to claim 17, wherein the fibers have a length ranging from 0.3 mm to 1 mm.

19. The composition according to claim 1, wherein the fibers have a cross section that is within a circle of diameter ranging from 2 nm to 500 μm.

20. The composition according to claim 19, wherein the fibers have a cross section that is within a circle of diameter ranging from 100 nm to 100 μm.

21. The composition according to claim 1, wherein the fibers have a length L and a diameter D such that the ratio L/D ranges from 1.5 to 2500.

22. The composition according to claim 21, wherein L/D ranges from 3.5 to 500.

23. The composition according to claim 22, wherein L/D ranges from 5 to 150.

24. The composition according to claim 1, wherein the fibers are present in an amount ranging from 0.5% to 20% by weight, relative to the total weight of the composition.

25. The composition according to claim 24, wherein the fibers are present in an amount ranging from 0.5% to 15% by weight, relative to the total weight of the composition.

26. The composition according to claim 25, wherein the fibers are present in an amount ranging from 0.5% to 8% by weight, relative to the total weight of the composition.

27. The composition according to claim 26, wherein the fibers are present in an amount ranging from 2% to 8% by weight, relative to the total weight of the composition.

28. The composition according to claim 1, wherein the fibers and the particles of elastomeric crosslinked organopolysiloxane are present in amounts such that the ratio of fibers to particles of crosslinked organopolysiloxane weight ratio ranges from 0.01 to 2.

29. The composition according to claim 28, wherein the ratio of fibers to particles of crosslinked organopolysiloxane weight ratio ranges from 0.03 to 1.

30. The composition according to claim 29, wherein the ratio of fibers to particles of crosslinked organopolysiloxane weight ratio ranges from 0.05 to 0.5.

31. The composition according to claim 30, wherein the ratio of fibers to particles of crosslinked organopolysiloxane weight ratio ranges from 0.05 to 0.2.

32. The composition according to claim 1, wherein the at least one additional powder is chosen from pulverulent dyestuffs and fillers.

33. The composition according to claim 1, wherein the at least one additional powder comprises at least one pulverulent dyestuff.

34. The composition according to claim 32, wherein the pulverulent dyestuff is chosen from at least one of pigments and nacres.

35. The composition according to claim 32, wherein the pulverulent dyestuff is chosen from at least one of titanium dioxide, zirconium oxides, cerium oxides, zinc oxides, iron oxides, chromium oxides, manganese violet, ultramarine blue, chromium hydrate, ferric blue, aluminium powder, and copper powder.

36. The composition according to claim 32, wherein the pulverulent dyestuff is present in an amount ranging from 0.5% to 60% by weight, relative to the total weight of the composition.

37. The composition according to claim 36, wherein the pulverulent dyestuff is present in an amount ranging from 0.5% to 50% by weight, relative to the total weight of the composition.

38. The composition according to claim 37, wherein the pulverulent dyestuff is present in an amount ranging from 0.5% to 40% by weight, relative to the total weight of the composition.

39. The composition according to claim 38, wherein the pulverulent dyestuff is present in an amount ranging from 1% to 30% by weight, relative to the total weight of the composition.

40. The composition according to claim 39, wherein the pulverulent dyestuff is present in an amount ranging from 3% to 25% by weight, relative to the total weight of the composition.

41. The composition according to claim 1, wherein the at least one additional powder comprises at least one filler.

42. The composition according to claim 41, wherein the at least one filler is chosen from talc, mica, silica, kaolin, polyamide powders, poly-β-alanine powders, polyethylene powders, tetrafluoroethylene polymer powders, lauroyllysine, starch, boron nitride, hollow microspheres of polyvinylidene chloride/acrylonitrile, hollow microspheres of acrylic acid copolymers, ethylene glycol dimethacrylate copolymer powders, lauryl methacrylate copolymer powders, silicone resin microbeads, precipitated calcium carbonate, magnesium carbonate, magnesium hydrocarbonate, hydroxyapatite, hollow silica microspheres, glass microcapsules, ceramic microcapsules, and metal soaps derived from organic carboxylic acids containing from 8 to 22 carbon atoms.

43. The composition according to claim 42, wherein the at least one filler is chosen from mica, polyamide powders, ethylene glycol dimethacrylate and lauryl methacrylate copolymer powders, and boron nitride.

44. The composition according to claim 41, wherein the at least one filler is present in an amount ranging from 0.5% to 40% by weight, relative to the total weight of the composition.

45. The composition according to claim 44, wherein the at least one filler is present in an amount ranging from 1% to 35% by weight, relative to the total weight of the composition.

46. The composition according to claim 45, wherein the at least one filler is present in an amount ranging from 5% to 30% by weight, relative to the total weight of the composition.

47. The composition according to claim 1, wherein the amount of the at least one additional powder ranges from 10% to 70% by weight, relative to the total weight of the composition.

48. The composition according to claim 47, wherein the total amount of the at least one additional powder ranges from 10% to 60% by weight, relative to the total weight of the composition.

49. The composition according to claim 48, wherein the total amount of the at least one additional powder ranges from 20% to 50% by weight, relative to the total weight of the composition.

50. The composition according to claim 1, wherein the particles of elastomeric crosslinked organopolysiloxane and the at least one additional powder are present in an amount such that the weight ratio of particles of crosslinked organopolysiloxane to at least one additional powder ranges from 0.4/1 to 2/1.

51. The composition according to claim 50, wherein the weight ratio of particles of crosslinked organopolysiloxane to at least one additional powder ranges from 0.4/1 to 1.5/1.

52. The composition according to claim 51, wherein the weight ratio of particles of crosslinked organopolysiloxane to at least one additional powder ranges from 0.4/1 to 1.3/1.

53. The composition according to claim 52, wherein the weight ratio of particles of crosslinked organopolysiloxane to at least one additional powder ranges from 0.6/1 to 1.3/1.

54. The composition according to claim 1, wherein the water is present in an amount ranging from 5% to 30% by weight, relative to the total weight of the composition.

55. The composition according to claim 54, wherein the water is present in an amount ranging from 10% to 25% by weight, relative to the total weight of the composition.

56. The composition according to claim 55, wherein the water is present in an amount ranging from 15% to 25% by weight, relative to the total weight of the composition.

57. The composition according to claim 56, wherein the water is present in an amount ranging from 20% to 25% by weight, relative to the total weight of the composition.

58. The composition according to claim 1, further comprising at least one polyol.

59. The composition according to claim 58, wherein the at least one polyol contains 2 to 20 carbon atoms.

60. The composition according to claim 59, wherein the at least one polyol contains 2 to 10 carbon atoms.

61. The composition according to claim 60, wherein the at least one polyol contains 2 to 6 carbon atoms.

62. The composition according to tclaim 58, wherein the at least one polyol is chosen from glycerol, propylene glycol, butylene glycol, pentylene glycol, hexylene glycol, dipropylene glycol, and diethylene glycol.

63. The composition according to claim 58, wherein the at least one polyol is present in an amount ranging from 1% to 20% by weight, relative to the total weight of the composition.

64. The composition according to claim 63, wherein the at least one polyol is present in an amount ranging from 3% to 15% by weight, relative to the total weight of the composition.

65. The composition according to claim 1, further comprising at least one cosmetic ingredient chosen from antioxidants, fragrances, preserving agents, neutralizers, surfactants, waxes, oils, sunscreens, vitamins, moisturizers, self-tanning compounds, and anti-wrinkle active agents.

66. The composition according to claim 1, wherein the composition has a hardness ranging from 0.07 N to 0.4 N and an elasticity EL ranging from 15% to 80%.

67. The composition according to claim 66, wherein the hardness ranges from 0.1 to 0.35 N.

68. The composition according to claim 66, wherein the elasticity EL ranges from 30% to 70%.

69. The composition according to claim 1, wherein the composition is a cosmetic composition.

70. The composition according to claim 1, wherein the composition is a makeup composition or care composition for keratin material.

71. The composition according to claim 70, wherein the keratin material is skin.

72. The composition according to claim 70, wherein the makeup composition is a product chosen from complexion products, eyeshadows, makeup rouges, concealer products, body makeup products, lip makeup products, nail makeup products, and hair makeup products.

73. The composition according to claim 72, wherein the complexion product is a foundation.

74. The composition according to claim 70, wherein the care composition is a product chosen from skincare products, skin matting products, antisun products for the skin, self-tanning compositions, and deodorant products.

75. A process for making up keratin material comprising applying to the keratin material a solid composition comprising water, solid particles of elastomeric crosslinked organopolysiloxane, fibers, and at least one additional powder.

76. The process according to claim 75, wherein the keratin material is skin.
